(19) United States
(12) Patent Application Publication
(10) Pub. No.: US 2025/0235714 A1
(43) Pub. Date: Jul. 24, 2025

(54) NON-INVASIVE LIGHT THERAPY FOR DRY EYE TREATMENT

(71) Applicant: MEDICAL QUANT USA INC., Solon, OH (US)

(72) Inventors: Douglas S. JOHNSON, Brownstown, MI (US); Maxim KANARSKY, Solon, OH (US); Thomas A. Kelly, Solon, OH (US); Darrell E. WHITE, Solon, OH (US)

(21) Appl. No.: 19/032,842

(22) Filed: Jan. 21, 2025

Related U.S. Application Data
(60) Provisional application No. 63/623,549, filed on Jan. 22, 2024.

(51) Int. Cl. A61N 5/06; G16H 20/13; G16H 20/40; G16H 40/63

(52) U.S. Cl. CPC A61N 5/0613; G16H 20/13; G16H 20/40; G16H 40/63; A61N 2005/0644; A61N 2005/0659; A61N 2005/0663

(57) ABSTRACT

Ocular surface disorders (OSDs), like dry eye disease, can have a variety of symptoms and underlying causes, so patients need personalized treatment plans. Treatment planning can include determining at least one treatment location for a non-invasive photoceutical based on at least one characteristic of the OSD. One or more photoceuticals, having one or more different parameters, can be designed based on the at least one treatment location and characteristic of the OSD to address interplay between at least two of inflammation, pain, and immune dysfunction related to the OSD. At least one treatment device (comprising at least one light source) can be selected to deliver the one or more photoceutical to the at least one treatment location to treat the OSD and at least one dose of the at least one photoceutical can be configured to be applied to at least partially treat the OSD.

### CROSS-REFERENCE TO RELATED APPLICATIONS

This application claims the benefit of U.S. Provisional Application No. 63/623,549, filed Jan. 22, 2024, entitled “NON-INVASIVE LIGHT THERAPY FOR DRY EYE TREATMENT”. This provisional application is hereby incorporated by reference in its entirety for all purposes.

### TECHNICAL FIELD

The present disclosure relates generally to ameliorating ocular surface disorders (OSDs) and, more specifically, to systems and methods for developing a patient-specific, non-invasive photoceutical-based treatment plan for a patient suffering from an OSD.

### BACKGROUND

Ocular surface disorders (OSDs) are a wide-ranging group of diseases, syndromes, and injuries that negatively affect the front layers of the eye that interface between a patient's eye and the external environment. The ocular surface includes the cornea, the conjunctiva, the tear film, and more. Any disruption to the harmonious function of the front layers of the eye can give rise to one or more of a host of OSDs (e.g., dry eye syndrome, conjunctivitis, corneal inflammation, abrasions, and ulcers, neoplastic growths, etc.). Symptoms of OSDs can range from temporary irritations, visual disturbances, dryness, and redness to severe pain and chronic, potentially vision-threatening conditions. Recognizing and properly addressing the myriad of symptoms of specific types of OSD is vital for maintaining normal visual function, eye health, and comfort and often requires professional evaluation and management. Current interventions are invasive (e.g., topical medications applied onto portions of the eye, surgical intervention, etc.) and often treat surface level symptoms and not the root causes of those symptoms.

### SUMMARY

The present disclosure relates to ameliorating an ocular surface disorder (OSD) with application of non-invasive photoceuticals. The systems and methods described herein provide personalized treatment plans, and application thereof, which apply one or more photoceuticals to one or more different locations in/on the body of a patient to treat both symptoms and underlying causes of the OSD.

In one aspect, the present disclosure can include a method for determining a treatment plan for a patient suffering from an OSD. The method includes: determining at least one treatment location based on at least one characteristic of the OSD; designing at least one photoceutical based on the at least one treatment location and the characteristic of the OSD, wherein the at least one comprises at least one parameter; selecting at least one treatment device to deliver the photoceutical to the at least one treatment location to treat the OSD, wherein the at least one treatment device comprises at least one light source; and configuring at least one dose of the at least one photoceutical to be applied to the at least one treatment location by the at least one treatment device to at least partially treat the OSD. The at least one dose of the at least one photoceutical is designed to address interplay between at least two of inflammation, pain, and immune dysfunction related to the OSD.

In another aspect, the present disclosure can include a method for treating an OSD. The method can include: determining a treatment plan to treat the OSD based on at least one characteristic of the OSD, the treatment plan comprising at least a first photoceutical and at least a second photoceutical; applying at least one first dose of the at least the first photoceutical to at least one primary location according to a first protocol; and applying at least one second dose of the at least the second photoceutical to at least one secondary location according to a second protocol. The combination of at least the first photoceutical applied according to the first protocol and the at least the second photoceutical applied according to the second protocol improves a treatment outcome of a patient suffering from the OSD by addressing interplay between at least two of inflammation, pain, and immune dysfunction based in the at least one characteristic of the OSD.

Also described herein are systems that can develop and/or apply the personalized, treatment plan including one or more photoceuticals to one or more different treatment locations of a patient suffering from a specific OSD.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a diagram showing example biological systems that may cause and/or affect a patient's ocular surface disorder (OSD);

FIG. 2 is a diagram showing an example of a system for developing a personalized treatment plan for a patient suffering from an OSD;

FIG. 3 is a diagram showing an example of the controller of FIG. 2;

FIG. 4 includes diagrams showing different example treatment locations that can be selected by the controller of FIG. 3;

FIG. 5 includes diagrams showing different example treatment devices that can be selected by the controller of FIG. 3;

FIG. 6 is a diagram showing an example of the system for developing a treatment plan for a patient suffering from another OSD and applying the treatment according to the treatment plan; and

FIGS. 7-9 are process flow diagrams of example methods for developing and applying a personalized treatment plan for an individual patient suffering from an OSD.

### DETAILED DESCRIPTION

### I. DEFINITIONS

Unless otherwise defined, all technical terms used herein have the same meaning as commonly understood by one of ordinary skill in the art to which the present disclosure pertains.

In the context of the present disclosure, the singular forms “a,” “an”, and “the” can also include the plural forms, unless the context clearly indicates otherwise.

As used herein, the terms “comprises” and/or “comprising” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “ocular surface” refers to the outermost surface of a patient's eye that is the primary interface between the patient's eye and the external environment. Components of the ocular surface include, but are not limited to the cornea, conjunctiva, and tear film. These components work together to maintain a stable, healthy environment for optimal vision while preventing discomfort.

As used herein, the term “anterior segment” generally refers to the front part of the eye that performs a function of focusing light on the retina. The anterior segment encompasses the ocular surface and includes the cornea, conjunctiva, iris, lens, aqueous humor, ciliary body, trabecular meshwork, Schlemm canal, ciliary muscle, zonules, and the like.

As used herein, the term “tear film” can refer to a thin layer of fluid that covers the ocular surface. Tear film functions to protect the eye from the environment (e.g., bacteria, debris, and the like), lubricate the surface of the eye during blinking, provide electrolytes to the cornea, and improve the eye's refractive power. The tear film has three key components: the mucin layer, the aqueous layer, and the lipid layer.

As used herein, the term “ocular surface disorder”, also referred to as “ocular surface disease” and “OSD”, refers to a range of conditions affecting at least a portion of a patient's anterior segment and/or tear film. The conditions can include, but are not limited to, common discomforting issues like Dry Eye Disease (DED), Meibomian Gland Dysfunction (MGD), blepharitis, and conjunctivitis to severe corneal inflammation, abrasions, and ulcers, keratitis, Allergic Eye disease, recurrent corneal erosion, neurogenic eye pain, and neoplastic growths, such as Ocular Surface Squamous Neoplasia. OSD can also include ocular growths like Pterygium and Pinguecula, dermatological conditions such as rosacea, as well as complications stemming from glaucoma medications, chemical burns, thermal burns, immunological disorders such as Mucous Membrane Pemphigoid and Sjogren's Syndrome, and neurological based eye pain (e.g., trigeminal neuralgia, etc.). In some instances, OSD can include one or more of post-refractive eye surgery discomfort, migraine pain, and the like.

As used herein, the term “dry eye disease”, also referred to as “DED” and “dry dye syndrome” or “DES”, refers to a multifactorial disease of the tears and/or ocular surface that results in mild to debilitating symptoms of discomfort, visual disturbance, and tear film instability with potential damage to the ocular surface, accompanied by increased osmolarity of the tear film and inflammation of the ocular surface. DED arises from factors such as aging, environmental influences, medication side effects, underlying medical conditions, or the like, and can result in ocular discomfort, redness, blurred vision, and a reduced quality of life.

As used herein, the term “treatment” refers to medical care given to a subject to heal or cure a medical condition, like an OSD, or a symptom of the condition. The terms “treatment” and “therapy” can be used interchangeably herein. In some instances, the treatment can be non-invasive and non-toxic.

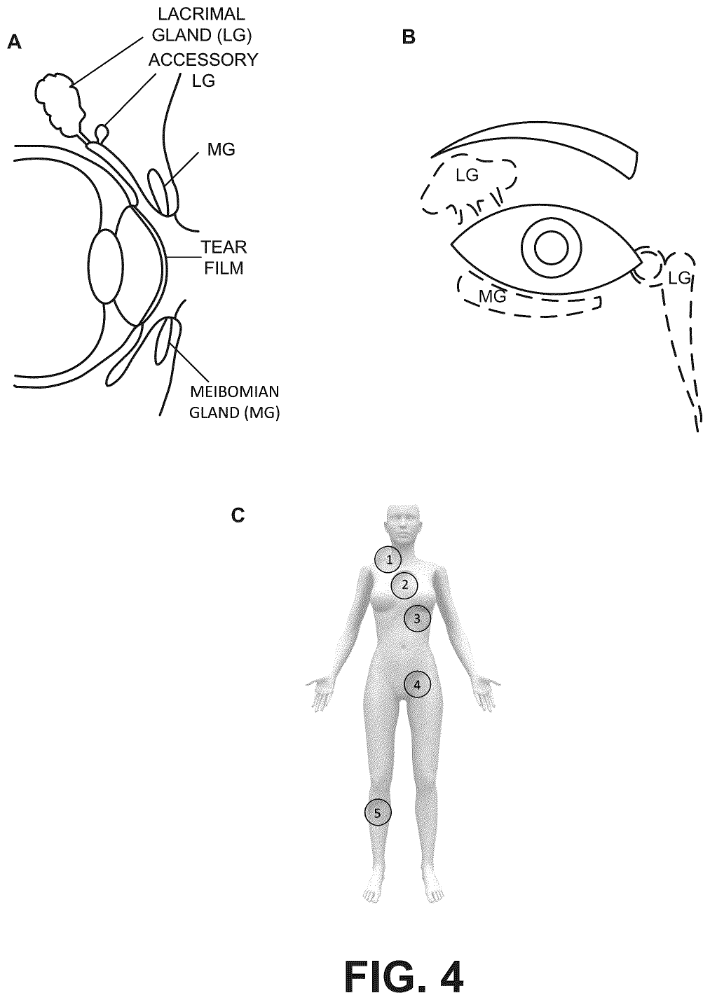

As used herein, the term “treatment location” refers to one or more locations on a surface of a patient's body where a photoceutical can be applied to treat OSD (see portions of FIG. 4, examples A, B, or C).

As used herein, the term “photoceutical” refers to a prescribed dose of light therapy to be delivered to the treatment location by a treatment device and used to change a function of a least a portion of a patient's body (e.g., by photobiomodulation to induce a phototherapeutic response using a drug-free, non-invasive treatment). In some instances, the light therapy can include one or more light signals (e.g., with the same or different wavelength, frequencies, intensities etc.). In other instances, the light therapy can include one or more light signals and one or more magnetic signals.

As used herein, the term “light therapy” refers to one or more light signals having one more parameters (e.g., wavelength intensity, power, timing, pulsation, and the like), intended to treat a certain condition. One or more light signals having one or more wavelengths can be combined to create a synergistic effect that can improve the percentage of available light at greater tissue depths. In some instances, the wavelengths can be within a wavelength range of 400-1100 nm. For example, the wavelengths can include at least one wavelength corresponding to the visible range of the electromagnetic spectrum (e.g., red light, 600-700 nm—reduces inflammation and triggers biochemical processes) and/or at least one wavelength corresponding to the near-infrared or infrared range (e.g., 700-1100 nm—deeper penetration, enhances circulation, reduces pain, affects cell membranes) of the electromagnetic spectrum. One example use of light therapy is photobiomodulation.

As used herein, the term “photobiomodulation”, also referred to as “PBM”, refers to a method for treating a portion of a subject's body with one or more light signals to induce a phototherapeutic response in cells within the portion of the subject's body. As an example, the portion of the subject's body can be a portion of a patient's ocular surface, but depending on the OSD, the portion of the subject's body can range beyond simply the ocular surface and may include one or more portions of the nervous system, the exocrine system, the muscular system, the immune system, the endocrine system, or the like.

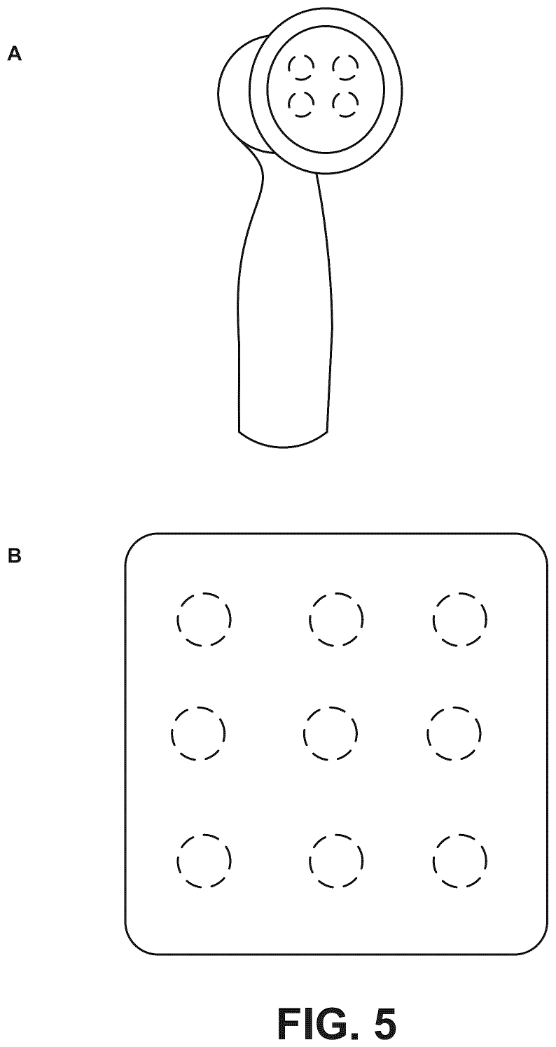

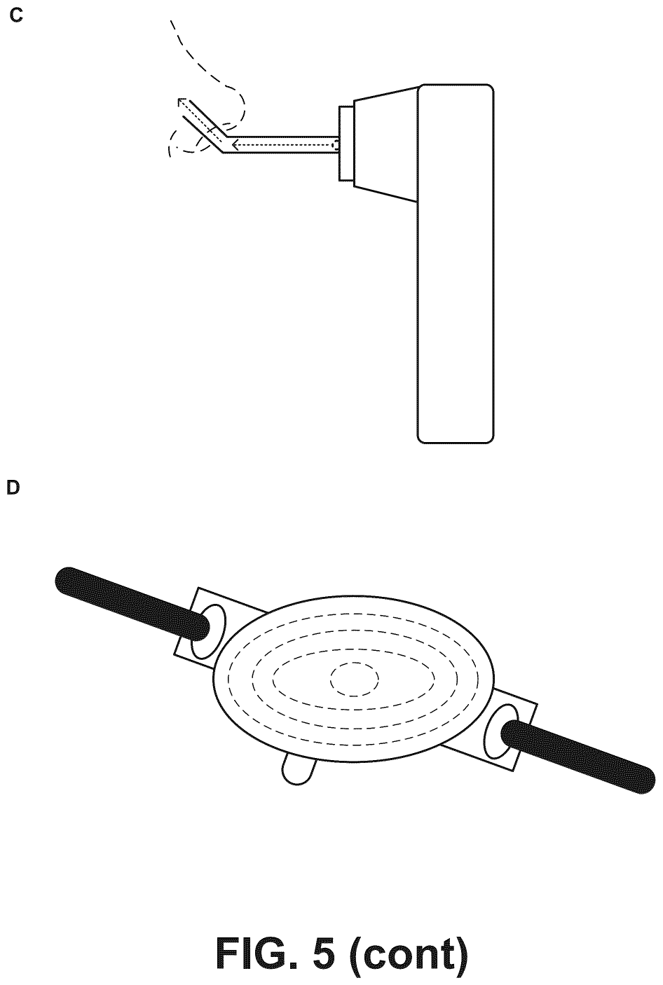

As used herein, the term “treatment device” refers to a device (see examples A, B, C, and D of FIG. 5) that can deliver at least PBMT to a treatment location. The treatment device can include at least a light delivery device and may include one or more light sources, super pulsed lasers, and/or magnets. The treatment device can include and/or can be in electrical communication (wired and/or wireless) with a controller that can provide power and/or parameter configurations (e.g., power(s), wavelength(s), intensit(ies), timing(s), light source choice(s), etc.) to the light delivery device of the treatment device.

As used herein, the term “light source” refers to a component of a light delivery device that can deliver one or more light signals having one or more wavelengths. A light source can be, for instance, a low-level laser source such as a laser diode, a light emitting diode (LED), an eye-safe laser, or the like. For example, the light source can be a low-level laser source (e.g., a laser diode) that generates coherent light. The low-level laser source can operate in a super pulsed mode that generates ultrashort pulses with a high peak power and minimal heat. As another example, the light source can be an incoherent light source, such as a traditional LED or light bulb. The incoherent light source can operate in a pulsed mode and/or a continuous mode. As another example, the light source can be a super pulsed laser. As another example, the light source can be an eye-safe laser.

As used herein, the term “super pulsed laser” refers to a light source that produces a wavelength of light at a high peak power for a very brief duration. Even though the pulse peaks at a high power level (e.g., up to 50,000 mW, up to 100,000 mW, up to 150,000 mW, up to 200,000 mW or the like), there are no thermal effects in the tissue due to the brevity of the pulse (e.g., billionth-of-a-second pulses, durations between 100 nanoseconds and 200 nanoseconds, or the like). The peak power is high compared to the average output power. By using a super pulsed laser, one is able to more effectively deliver higher densities of light energy into the tissue without associated deleterious thermal effects. A super pulsed laser can be, for example, a super pulsed infrared laser (905 nm) using Gallium Aluminum Arsenide (GaAS) diodes.

As used herein, the term “eye-safe laser” refers to a laser that emits light signals having wavelengths that are absorbed or mostly absorbed by the cornea and lens of the eye, preventing the light signals from reaching the retina and causing damage to the highly sensitive retina. An eye-safe laser can, for example, emit wavelengths longer than 1.4 μm. Examples of eye-safe lasers can include but are not limited to erbium lasers and erbium-doped fiber amplifiers emitting in the 1.5 μm spectral range, thulium lasers around the 2-μm range, semiconductor lasers, and lasers with optical parametric oscillator sources.

As used herein, the term “dose” can refer to a quantity of a treatment taken at a particular time.

As used herein, the terms “patient” and “subject” can be used interchangeably and refer to any warm-blooded organism that can suffer from an OSD including, but not limited to, a human being, a pig, a rat, a mouse, a dog, a cat, a goat, a sheep, a horse, a monkey, an ape, a rabbit, a cow, etc.

### II. OVERVIEW

A patient's ocular surface can function harmoniously when all of its components work together to maintain a stable, healthy environment for optimal vision while preventing discomfort. The components of the ocular surface can include (but are not limited to) the cornea and conjunctiva, as well as tear film. Any disruption to this harmonious function, due to the external environmental factors, genetics, bacteria, viruses, etc., can give rise to one or more of a host of ocular surface disorders (OSDs). OSDs are a wide-ranging group of diseases, syndromes, and injuries that can have mild, temporary affects or severe and chronic affects with long-term vision complications. For example, OSDs can include dry eye syndrome, conjunctivitis, corneal inflammation, abrasions, and ulcers, neoplastic growths, and the like. Symptoms of OSD can include temporary irritations, visual disturbances, dryness, and redness, but may be severe, and can include chronic pain and partial or full vision loss. Current interventions are invasive (e.g., topical medications applied onto portions of the eye, surgical intervention, etc.) and often treat surface level symptoms and not the root causes of those symptoms. Recognizing and properly addressing the myriad of symptoms of specific types of OSD is vital for maintaining normal visual function, eye health, and comfort and often requires professional evaluation and management.

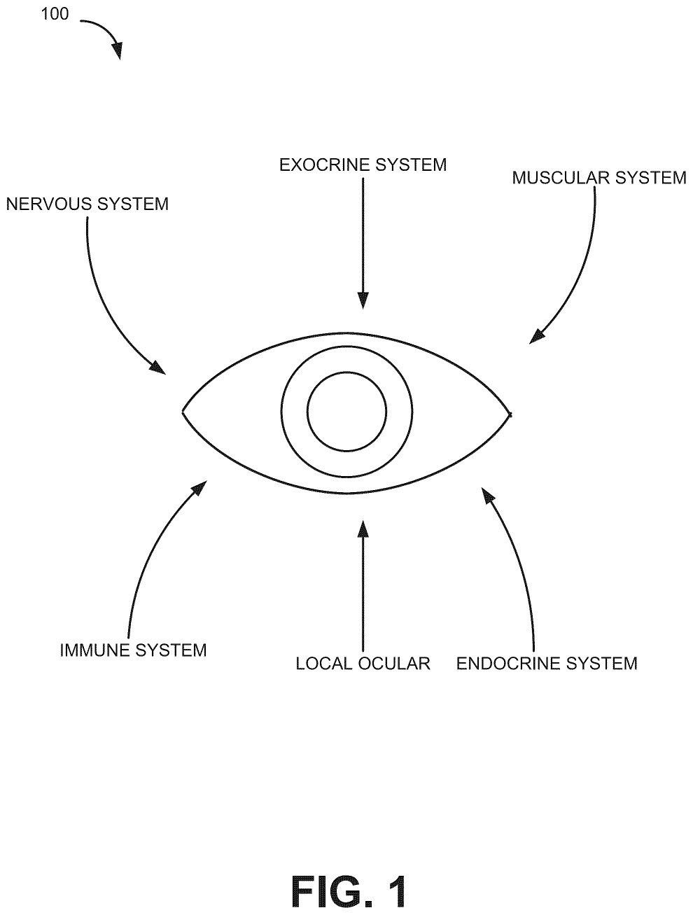

Described herein are systems and methods for developing a personalized non-invasive photoceutical based treatment plan for a patient suffering from an OSD. This can reduce (and in some instances eliminate) the requirement for continuous professional evaluation/management and invasive interventions. The systems and methods recognize that there a variety of OSDs that include a variety of overlapping symptoms with diverse causes that can be non-invasively treated in a personalized manner for each person suffering an OSD. For each patient, treatment plans can include determining at least one treatment location, a personalized photoceutical combinations and dosages, and a type of treatment device to best apply the photoceutical(s) to the at least one treatment location, all based on the specific OSD characteristics of the patient. OSD characteristics can include, but are not limited to, symptoms, locations of symptoms, underlying injuries, diseases, genetic disorders, known environmental causes, and the like. The treatment locations can be a portion of the ocular surface (e.g., see FIG. 4, element D) or localized eye structures (e.g., tear glands/ducts) (see FIG. 4, elements A and B) and/or one or more body parts distant from the eye (see FIG. 1 and FIG. 4, element C). The photoceuticals can be designed to address interplay between at least two of inflammation, pain, and immune dysfunction related to the OSD based on the treatment location(s) and the characteristic of the OSD.

### III. SCIENTIFIC FEATURES

### Ocular Surface Disorders (OSDs)

The ocular surface is a part of a patient's visual functional unit and has recently been recognized as an immunological unit capable of responding to both external and internal stimuli. Recognizing and addressing Ocular Surface Disorders (OSDs) is vital for maintaining normal visual function, eye health, and comfort. Emerging understanding of the ocular surface as more than just a component of the visual functional unit underscores the complex and interconnected nature of ocular health. The eye not only facilitates vision, but also actively engages in responses with other parts of the body to maintain well-being of the ocular surface. As shown in FIG. 1, one or more components of the nervous system, exocrine system, muscular system, immune system, and/or the endocrine system, as well as components local to and/or within the eye (local ocular), can affect the well-being of the ocular surface.

OSDs can arise when the well-being of the ocular surface is not maintained. OSDs are a comprehensive category encompassing a diverse spectrum of conditions affecting at least a portion of a patient's anterior segment (e.g., the cornea, conjunctiva, related surfaces, and the like) and/or the tear film. OSDs can include, but are not limited to, common discomforting issues like Dry Eye Disease (DED), Meibomian Gland Dysfunction (MGD), blepharitis, and conjunctivitis (pink eye) as well as more serious issues like corneal inflammation, abrasions, and ulcers, keratitis, Allergic Eye Disease, recurrent corneal erosion, neurogenic eye pain, and neoplastic growths, such as Ocular Surface Squamous Neoplasia. OSD can also include ocular growths like Pterygium and Pinguecula, dermatological conditions such as rosacea, as well as complications stemming from glaucoma medications, chemical burns, thermal burns, immunological disorders such as Mucous Membrane Pemphigoid and Sjogren's Syndrome, and neurological based eye pain (e.g., trigeminal neuralgia, etc.). In some instances, OSDs can also include post-refractive eye surgery discomfort (specifically following procedures like laser-assisted in situ keratomileusis (LASIK), small incision lenticule extraction (SMILE), and photorefractive keratectomy (PRK), for example).

DED is an example of an OSD. DED is a significant ocular condition characterized by discomfort and disruptions in the tear film. More specifically, DED is a multifactorial disease of the tears and ocular surface that results in symptoms of discomfort, visual disturbance, and tear film instability with potential damage to the ocular surface, accompanied by increased osmolarity of the tear film and inflammation of the ocular surface. Generally, the tear film plays a vital role in maintaining eye health and clarity. A well-functioning tear film consists of three key components: the mucin layer, aqueous layer, and lipid layer, all of which are essential for ocular comfort and visual acuity. DED can manifest in various forms, with symptoms ranging from mild irritation to chronic and debilitating irritation. The condition arises from factors such as aging, environmental influences, medication side effects, or underlying medical conditions. In many cases, DED can result in ocular discomfort, redness, blurred vision, and a reduced quality of life for affected individuals. Traditional treatments for dry eye syndrome primarily involve the application of artificial tears, topical medications, or, in severe cases, invasive surgical procedures. These treatments often have limitations in terms of effectiveness, and patients may experience difficulties with adherence.

Non-Sjögren's DED is a subtype of DED that primarily results from lacrimal dysfunction and lacks the overt signs of systemic autoimmunity associated with Sjögren's syndrome. The most prevalent form of Non-Sjögren DED is age-related dry eye, which is characterized by decreased tear volume and flow, elevated tear osmolarity, reduced tear film stability, and changes in the composition of Meibomian lipids. Other common triggers for DED, which can contribute to the chronic cycle of the condition, can include systemic medications that inhibit tear production, hormonal factors (with lower androgen levels potentially promoting ocular surface inflammation), low humidity, environments with constant airflow leading to increased tear evaporation, prolonged use of preserved eye drops, contact lens wear, and refractive eye surgery. Understanding these various causes and which ones affect a given patient is crucial for effective management and treatment of DED.

### Photoceutical

OSDs, like DED discussed above, are multifaceted conditions that have historically posed challenges due to limited treatment options, which range from artificial tears to invasive surgeries. Current treatments are ineffective and/or fully invasive. Described herein is a non-invasive, non-pharmacological, and patient-friendly alternative treatment option that delivers one or more photoceuticals,) which captures the potential of at least light therapy (also referred to as photobiomodulation (PBM)) and in some cases a combination of light and magnetic therapy. The one or more photoceuticals can also be used for diagnosis, management, or the like in addition or alternatively to treatment. Photoceuticals, including photobiomodulation, provide a multifaceted, patient-focused approach that overcomes existing limitations related to current treatments and provides improved patient outcomes. It should be understood that photoceuticals can be used as a standalone treatment and/or as a complementary therapy in conjunction with one or more other medical interventions (e.g., surgical intervention and/or topical medication application).

The non-invasive treatment option includes applying one or more photoceuticals to one or more treatment locations on and/or in a patient's body according to a prescription to treat an OSD. As an example, the one or more treatment locations can be associated with a patient's eye. As another example, the one or more treatment locations can include other parts of the patient's body (see, e.g., FIG. 1 for bodily systems that may work in conjunction with the ocular surface) to maintain well-being of the ocular surface. The photoceutical can be delivered with a treatment device that can deliver a predefined amount of light therapy to the one or more treatment locations according to a prescription. The light therapy can include one or more light signals having one more parameters (e.g., wavelength, intensity, power, timing, pulsation, and the like), intended to treat a certain condition. In some instances, the light therapy can combine a plurality of light signals into a PBM signal. However, the light therapy may simply include one or more light signals that may be combined in different ways.

Notably, the use of carefully selected wavelengths of light, delivered by eye-safe LEDs and heat-free light sources, distinguishes from traditional methods, placing a strong emphasis on patient safety and comfort. The non-invasive nature minimizes the risk of complications and discomfort, ultimately enhancing patient adherence to treatment. For ocular light delivery, safety considerations are paramount to avoid thermal damage, preserve ocular tissue, and preserve sight. Patient comfort is also an important consideration as the eye is an extremely sensitive organ. However, light delivered to areas that are not the eye can be conducted without the safety considerations required for ocular applications.

Generally, the light signal(s) work by promoting cellular respiration normalization, immune modulation, enhanced circulation, and addressing neuropathic pain, this approach rejuvenates ocular tissues, reduces inflammation, and expedites healing. The light has been shown to have a modulatory effect promoting healing based on the principle that certain molecules in living systems (cellular chromophores, chiefly mitochondria) absorb photons (in other words, harness light energy) and trigger signaling pathways in response to light initiating a cascade of photochemical reactions that bestow a multitude of benefits crucial for efficient tissue repair and regeneration. While not wishing to be bound by theory, there is strong evidence to suggest that one of the basic cellular tasks mechanisms of photobiomodulation therapy (PBMT) (which is an example of a photoceutical) is the acceleration of electron transfer by electromagnetic radiation in the visible and near infrared region of the spectrum, via the modulation of cytochrome c-oxidase (“CCO”) activity in cells. CCO is the primary photo acceptor of visible to near infrared light energy and is the enzyme responsible for catalyzing oxygen consumption in cellular respiration and for the production of nitric oxide under hypoxic conditions. High-energy electrons are passed from electron carriers through a series of trans-membrane complexes (including CCO) to the final electron acceptor, generating a proton gradient that is used to produce adenosine triphosphate (ATP), the body's energy currency (fueling a wide array of cellular functions essential for tissue repair). The application of light directly results in ATP production and electron transport. In short, the application of PBMT can increase ATP production, down-regulate cellular respiration modulated by nitric oxide (NO), and promotes the metabolism of oxygen, while increasing the production of reactive oxygen species (ROS). The heightened ATP availability empowers cells involved in healing of OSD of the eye with the necessary energy reservoirs to drive the reparative process.

The improved ATP production is intimately linked to elevated cellular respiration. Enhanced cellular respiration not only augments the efficiency of energy conversion but also fuels a more robust metabolic environment. This accelerated metabolism accelerates the synthesis of essential molecules, such as proteins and enzymes, pivotal for cell growth and tissue repair. Consequently, the overall healing process is expedited, leading to quicker restoration of the ocular surface.

The cascade of photochemical reactions initiated by application of the one or more light signals extends beyond energy metabolism. These reactions can stimulate cellular signaling pathways that govern various aspects of cellular behavior. For instance, the activation of signaling molecules like reactive oxygen species (ROS) can trigger beneficial responses, including cellular proliferation and the modulation of inflammatory processes. This plays a pivotal role in creating an environment conducive to healing while simultaneously reducing the risk of chronic inflammation that could impede recovery. The comprehensive enhancement of tissue repair orchestrated by PBMT is underpinned by its multi-faceted approach. Not only does it increase ATP production and cellular respiration, but it also bolsters the production of essential molecules, influences cellular signaling, and maintains a balanced inflammatory response, fostering an environment conducive to efficient healing.

Moreover, application of the one or more light signals exhibits a remarkable capacity to finely modulate immune responses, including the intricate control of interferon gamma (IFN-γ) levels. Toll-like receptors (TLRs) in ocular surface disease play a pivotal role in recognizing and responding to pathogens and inflammatory signals on the ocular surface. These receptors detect pathogen-associated molecular patterns (PAMPs) and damage-associated molecular patterns (DAMPs) in the eye, triggering immune responses. In ocular surface diseases like dry eye syndrome and conjunctivitis, TLRs can become activated, leading to inflammation and immune responses, contributing to disease pathology.

Through the precise application of specific wavelengths, laser light can penetrate tissue and interact with immune cells, triggering a cascade of responses. Notably, laser therapy demonstrates the unique ability to both up-regulate and down-regulate IFN-γ production. In situations requiring an immune boost, such as combating infections, laser therapy enhances IFN-γ synthesis, empowering immune cells for more effective pathogen defense. Conversely, in scenarios marked by IFN-γ overexpression, like autoimmune diseases, laser therapy curtails excessive production, thereby mitigating immune hyperactivity and dampening harmful inflammatory processes. This dual modulatory effect highlights laser therapy's potential as a versatile tool for immune regulation, offering a tailored approach to address diverse immune-related conditions.

The precision in applying photoceutical(s) showcases impressive capabilities for fine-tuning immune responses, notably through the modulation of inflammatory marker levels. This therapeutic technique interacts directly with immune cells, setting off a cascade of reactions. Most notably, laser therapy possesses the unique ability to both augment and suppress IFN-γ production as required. For example, laser therapy can boost IFN-γ synthesis in scenarios demanding immune response reinforcement, such as when combatting infections. Conversely, in situations characterized by excessive IFN-γ production, such as in autoimmune diseases, laser therapy effectively curbs this surplus, alleviating immune hyperactivity and mitigating detrimental inflammatory processes. This dual modulatory capacity places laser therapy as a versatile tool for immune regulation, providing a tailored approach to address a wide spectrum of immune-related conditions, particularly in the context of dry eye syndrome and other OSDs.

There is a pivotal role played by T lymphocytes, notably CD4+ T cells, in the initiation of pathological changes observed in dry eye syndrome (DES and also referred to as DED). This understanding contributes significantly to our knowledge of the condition's pathogenesis. Tear samples from individuals with DES have been analyzed to identify pertinent biomarkers, which include TNF-alpha, interleukin 6 (IL-6), IL-8, and IL-17. These markers consistently display elevated levels in the tear fluid of affected individuals, providing vital insights into the underlying inflammatory mechanisms associated with this ailment. The application of photoceuticals such as photobiomodulation have been demonstrated to influence the expression of key proteins, including TNF-alpha, IL-8, TGF-beta, IL-1, INF gamma, IL-6, and PGE-1. These markers may be part of the complex inflammatory landscape implicated in the development and progression of dry eye syndrome, revealing potential avenues for therapeutic intervention.

Healing is intricate and heavily reliant on a well-coordinated interplay of various biological factors. Among these factors, the stimulation of angiogenesis through the application of specific wavelengths of light stands out as a pivotal mechanism that significantly influences the healing process. Angiogenesis, the growth of new blood vessels from existing ones, is a fundamental process in tissue repair and regeneration. Certain wavelengths of light have been scientifically demonstrated to induce and accelerate angiogenesis in the affected area. This phenomenon is of paramount significance due to its multifaceted impact on the healing environment. The formation of new blood vessels brings about a host of benefits that collectively create an optimal milieu for tissue repair. One of the most immediate advantages is the enhanced delivery of oxygen and nutrients to the ulcerated site. Oxygen is a critical component for cellular metabolism and energy production, both of which are essential for the cells involved in the regenerative process. Furthermore, the increased supply of nutrients facilitates the rebuilding of damaged tissue components, aiding in the restoration of the corneal structure. Additionally, the new blood vessels act as conduits for immune cells, which play an indispensable role in combatting infections and orchestrating the intricate processes of tissue repair. By fostering an increased presence of immune cells at the ulcerated site, the immune response becomes more efficient and effective, thereby accelerating the clearance of pathogens and debris. Furthermore, the enhanced blood circulation resulting from angiogenesis promotes the removal of waste products and toxins from the site of injury. This detoxification process further contributes to the establishment of a healthier environment for cellular regeneration and healing. By promoting the growth of new blood vessels, the therapy sets in motion a cascade of positive effects—improved oxygenation, enhanced nutrient supply, increased immune cell presence, and efficient waste elimination.

Improved blood circulation facilitates the removal of waste products and toxins from the injury site. This detoxification mechanism plays a pivotal role in creating a more favorable environment for cellular regeneration and recuperation. Delivery of oxygen and essential nutrients to the affected site is enhanced, fostering an augmented presence of immune cells at the injury site, thereby rendering the immune response more efficient and effective, advancing the overall healing process.

Moreover, understanding the intricate connection between the sphenopalatine ganglion (SPG) and OSD is pivotal in the diagnosis and management of many ocular conditions. Therapies directed at modulating the autonomic nervous system, including interventions targeting the SPG, may hold promise in the treatment of conditions involving tear production, inflammation, and vascular changes on the ocular surface. This interplay hinges on the autonomic nervous system's role in regulating tear production, blood flow, and inflammation within the eyes. Comprising both sympathetic and parasympathetic branches, the parasympathetic division, housing the SPG, emerges as particularly significant in the context of ocular health and ocular surface diseases. By recognizing this relationship, healthcare providers can better tailor treatments and interventions, addressing not only the local manifestations of OSDs but also the underlying neural and vascular mechanisms that contribute to the conditions.

An overactive SPG can result from a variety of factors, including underlying neurological conditions and inflammation. The SPG's role in pain signaling can contribute to its dysfunction, leading to symptoms like excessive tearing, headaches, and facial pain. Managing an overactive SPG typically involves addressing the underlying cause and may include medication, nerve blocks, or other therapeutic approaches to alleviate the associated symptoms. Targeting the SPG aims to reduce neuronal excitation, thereby regulating tear production and ocular surface health. By reducing excitation, this approach may promote improved tear production and alleviate dry eye symptoms.

### IV. SYSTEMS

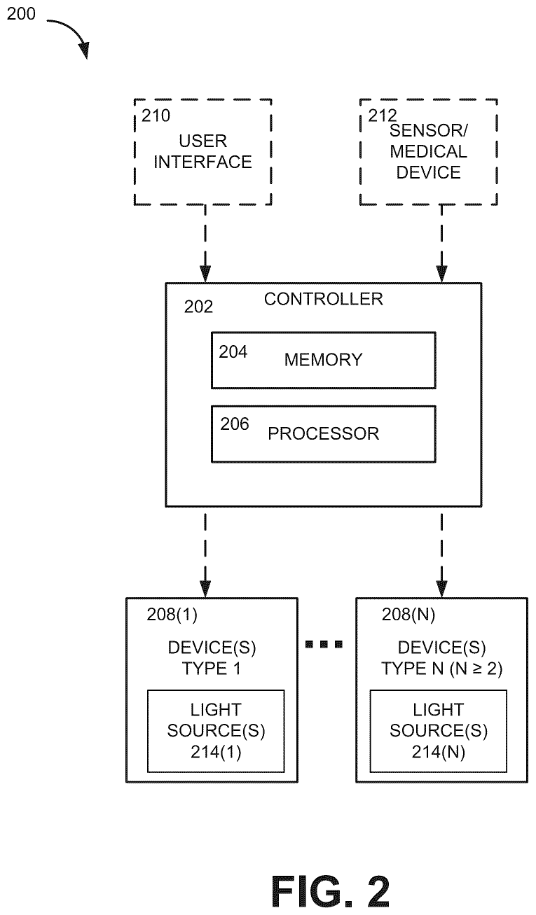

Ocular Surface Disorders and Diseases (both referred to herein as OSDs) can have a number of different causes, manifestations, and symptoms in different patients. Traditional treatments do not work for every patient with an OSD. For example, traditional treatments (e.g., topical application of pharmaceuticals directly to the eye, manual gland palpitations, and even some surgeries) may mask or alleviate a symptom of an OSD without treating an underlying cause. As shown in FIG. 1, OSDs can be caused by disorders, deficiencies, injuries, or the like in one or more of the nervous system, the exocrine system, the muscular system, the immune system, the endocrine system, or the local ocular area). As shown in FIG. 2, a system 200 can develop a personalized treatment plan that can be unique for different patients suffering from OSD. Conditions, such as medical history, prior diagnosis, symptoms, symptom locations, symptom severity, and the like, of the OSD can be input into the system 200 and the system can determine and provide the unique treatment plan to the patient based on an analysis of the various inputs. The treatment plan can include one or more photoceuticals of one or more doses to be applied to one or more treatment locations (located on one or more surfaces of the patient's body) for a certain time period and/or frequency. The one or more treatment locations can be one or more parts of the patient's body related in some way to the OSD (e.g., at and/or proximal to the eye or distal from the eye but connected to a cause of the OSD). Each treatment plan can offer precise, patient-friendly treatment for the OSD (also referred to as a “personalized treatment plan”) including which treatment device to use for which dose and treatment location, reducing complications associated with treating the OSD, minimizing patient discomfort, easing patient concerns, and enhancing safety and patient compliance.

The personalized treatment plan can be developed for each patient by a controller 202 based on data that is input from a user interface 210 and/or a sensor or medical device 212. The controller 202 can be in wired and/or wireless communication with the user interface 210 and/or the sensor or medical device 212. The controller 202 can include at least a non-transitory memory (memory 204) and a processor 206. While not shown, it should be understood that the controller 202 may also include a display, a generator, an internal power source, a connection for external power, and the like. It should also be understood that even though the controller is shown in FIG. 2 as a single device, the controller 202 can be embodied as a single device and/or a plurality of connected devices (e.g., in electrical communication, or the like). The controller 202 can be, for example, a computer, a smartphone, a tablet, a specially designed device, or the like. In some instances, at least a portion of the data (e.g., patient information (e.g., age, gender, weight, height, history, etc.), diagnosis, symptoms, etc.) can be input by a user (e.g., a patient, a medical professional, an assistant, or the like) via a user interface 210. The user interface 210 can include, for example, a keyboard, a mouse, one or more buttons, knobs, and/or switches, a touch screen, a microphone, a separate device including another controller like a smart phone, computer, or tablet in wireless communication with the controller 202, or the like. In other instances, at least another portion of the data (e.g., historical symptoms, current symptoms, general patient health information (e.g., heart rate, oxygen level, glucose count, etc.), compliance with light application/following prescription, use of the device, prior use of the device, etc.) can be input be one or more sensors and/or medical devices 212 (associated with the patient and/or associated with the patient's medical records or electronic health record (her)). The one or more sensors and/or medical devices 212 can be or can include, for example, an oxygen sensor, a heart rate sensor, a blood pressure sensor, an intraocular pressure sensor, a tear film sensor, or the like. In still other instances, still another portion of the data (e.g., personal data, etc.) can be input by a user and/or by mining other computer programs/computer-related information. It should be noted that any portion of the data can be input in any manners described herein and is not limited to user interface 210 or sensor(s) and/or medical device(s) 212 specifically and is not limited to the examples provided herein.

The controller 202 can include at least a memory (e.g., a non-transitory memory) 204 that can store instructions, data, etc. and a processor 206 that can access the memory 204 and execute the instructions, use the data, etc. In this example, the instructions and data are related to developing the treatment plan for the patient (but the instructions/data can be different). As illustrated, the memory 204 and the processor 206 can be individual devices. It should be understood that functionality of the non-transitory memory 204 and processor 206 can be embodied within a single device (e.g., a microprocessor). The at least one processor 206 can execute the instructions for the controller 202 and/or one or more connected device (e.g., one of devices 208(1)-208(N)) to at least perform one or more actions (e.g., determine at least a portion of the treatment plan, apply a portion of a treatment plan, etc.).

The controller 202 can output at least a portion of the personalized treatment plan to one or more devices 208(1)-208(N) each including at least one light source 214(1)-214(N) (N is a number of unique devices necessary to deliver the treatment to one or more treatment locations such that the personalized treatment plan can be executed at the treatment location(s) for the patient). Examples of the different types of treatment devices 208(1)-208(N) that can deliver the treatment to the treatment location(s) for the patient are shown and discussed in detail in connection with FIG. 5 (however, these are only examples for explanatory purposes and other types and configurations of treatment devices should be understood). In some instances, the controller 202 can be embodied within the at least one treatment device 208(1)-208(N)—in the handle of FIG. 5, elements A or C, for example. In other instances, the controller 202 can be at least partially in a separate device (at least a portion can be within the at least one treatment device 208(1)-208(N) and at least a portion within another device, in some instances, like in FIG. 5, element B). In still other instances, the controller 202 can be entirely separate (in different devices, such as the example shown in FIG. 5, element D) than the at least one treatment device 208(1)-208(N) and can be and wireless and/or wirelessly connected to the at least one treatment device.

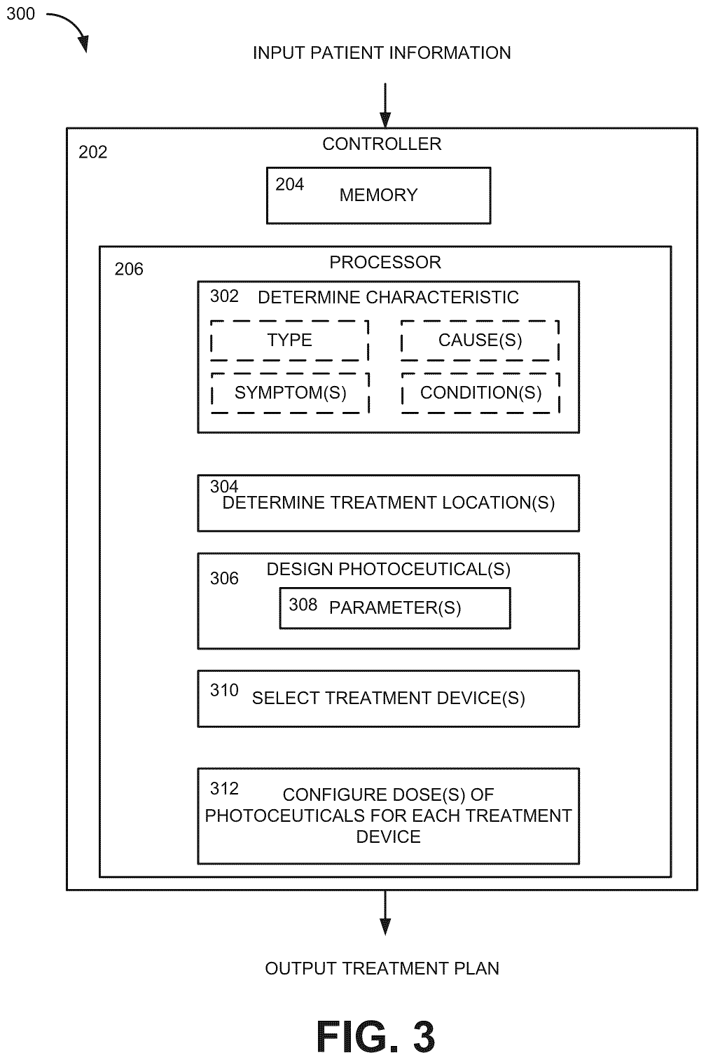

FIG. 3 is a diagram showing an example of the controller 202 of FIG. 2 determining a personalized treatment plan for a patient. The processor 206 can execute the instructions and utilize data stored in the memory 204 and/or patient information input into the controller 202 (e.g., from the user interface 210 and/or sensor(s) and/or medical device(s) 212). At least one characteristic 302 of the OSD can be determined from the data in memory 204 and/or the input patient information. The at least one characteristic 302 can include the type of OSD (e.g., aqueous-deficient dry eye, evaporative dry eye, etc.), one or more causes of the OSD (e.g., injury, age, underlying disease, genetic factor, etc.), one or more symptom(s) (including location, severity, and the like), one or more condition(s) (e.g., dry eye syndrome, conjunctivitis, keratitis, severe corneal inflammation, abrasions, ulcers, neoplastic growths, etc.), or the like. The one or more characteristics can be determined by a combination of the input and/or saved data of a particular patient. In some instances, patient data can be compared to known population data for various types of OSDs for a similar group of people, personal baselines, or the like. One or more treatment locations 304 (on and/or in the patient's body) can be determined based on one or more of the at least one characteristic. The treatment locations 304 can be anatomical targets that can include (but are not limited to): the tear film and/or origination points for the tear film (e.g., lacrimal gland(s)), meibomian gland(s), etc.), ocular surface epithelium, lymphoid organs, sensory nerves or the like. The treatment locations 304 can be any locations where application of at least one photoceutical can reduce inflammation, reduce pain, regulate immune factors/response, or the like (other examples shown in FIG. 1). In some instances, at least a portion of the treatment location(s) 304 can be on the patient's eye affected by OSD (e.g., see FIG. 4, elements A and B). In other instances, at least a portion of the treatment location(s) 304 can be remote from the patient's eye affected by OSD (e.g., see FIG. 4, element C).

It should be noted that at least the portion of the treatment location(s) 304 can be one or more locations shown in FIG. 4, elements A and B, chosen based on at least one characteristic. FIG. 4, element C, shows example locations where the photoceutical can be delivered remote from the patient's eye to one or more the bodily systems that can be treated (e.g., one or more factors to reduce inflammation, one or more sensory nerves, one or more lymph nodes, or the like). For example, one treatment location can be one of the meibomian gland(s) and another treatment location can be the lacrimal gland if one of the at least one characteristic is tear film dysfunction. Meibomian glands produce an oily substance called meibum that coats the surface of the eye and prevents tears from evaporating. Lacrimal glands produce tears. In another example, inflammation of the ocular surface can have the ocular surface epithelium as one treatment location (e.g., reduce local inflammatory molecules) and/or one or more lymphoid organs (e.g., white pulp, lymph nodes (1,4), thymus gland (2), spleen (3), bone marrow (5), skin (dorsal and ventral), etc.) and/or lymph nodes (e.g., regulate immune response for global inflammation reduction). It should be understood that these examples are for illustrative purposes only and not meant to be limiting.

Referring again to FIG. 3, one or more photoceuticals can be designed 306 based on the one or more treatment locations and/or the at least one characteristic. Designing the one or more photoceuticals 306 can include selecting one or more parameters 308 for each of the one or more photoceuticals. The one or more parameters 308 can include at least one of: at least one wavelength of light capable of photobiomodulation, at least one magnetic field strength, at least a power or energy per dose, a pulsation characteristic (e.g., continuous, pulsed, superpulsed, etc.), etc. The one or more parameters 308 can include at least one of the same or all different parameters for each of the one or more photoceuticals depending on the characteristics and treatment locations. For instance, different wavelengths or powers of light may be used to treat the local ocular area (e.g., ocular surface, meibomian glands, lacrimal glands, etc.) compared to one of the lymphoid organs and/or a neural target. In another instance, one or more parameters 308 of the one or more photoceuticals can be determined based on a severity of a symptom and/or a type of the OSD (e.g., is it aqueous-deficient dry eye or evaporative dry eye). Then, one or more treatment devices can be selected 310 based on the one or more of the characteristics 302, one or more of the treatment locations 304, and the one or more designed photoceutical 306. The system 300 can choose which of the available devices can best apply the designed photoceutical 306 to the at least one determined treatment location 304 for the determined characteristics 302. Examples of the types of treatment devices are shown and described in greater detail with respect to FIG. 5. According to one example, the one or more photoceuticals can include a photobiomodulation therapy that includes at least light of at least one wavelength emitted by at least one light source. One or more dose(s) of photoceuticals can be configured 312 to be delivered for each treatment device based on one or more of the characteristics 302, one or more of the treatment locations 304, one or more of the treatment devices 312, etc. The one or more dose(s) of the at least one photoceutical can be designed specifically for the patient to address interplay between at least two of inflammation, pain, and immune dysfunction related to the OSD. For example, the one or more doses 312 can include a number of applications of the at least one photoceutical, a length of time of each application of the at least one photoceutical, an exposure time, and/or a time between applications of the at least one photoceutical to the at least one treatment location, or the like.

As noted, FIG. 5 illustrates different example treatment devices that can be selected by the controller 202 of FIGS. 2 and 3. Different treatment devices can be selected based on the treatment location and one or more characteristics of the user. The handheld treatment device of FIG. 5, element A and the pad-like device of FIG. 5, element B (for larger treatment locations) can be used when the treatment location is remote from the eye (e.g., held or positioned over one or more lymphoid organs, neural locations, or the like) but may also be used to deliver light to the eye and/or surrounding area (e.g., glands, facial nerves, etc.) (e.g., the hand held device can be direct to and/or across the eye or directed to the gland areas above and/or below the eye, the pad like device can be placed over one or both eyes and at least a portion of the face (e.g., to reach gland areas and/or nerves). The nasal handheld device of FIG. 5, element C can be used to deliver light through the nose (or other opening into the body like the ear) to be closer to one or more neural targets such as the trigeminal nerve. The nasal handheld device can include in some instances a light pipe to help direct the light through the nasal cavity to be closer to the nerve (e.g., to lessen attenuation). The eye patch device shown in FIG. 5, element D can be used to deliver light to the eye (e.g., anterior segment, tears, etc.) or glands near the eye. The eye patch device can be desirable in cases with longer treatment times, more severe symptoms, less patient mobility, or the like.

The treatment device shown in FIG. 5, elements A, B, C, and D can be configured to deliver one or more wavelengths of light (e.g., from lasers, such as eye-safe lasers, LEDs, or the like) to offer a non-invasive, patient-friendly solution. They offer a distinct advantage over devices and/or pharmaceuticals that need to be applied directly to the eye and/or under the eyelids as no direct contact with the eye or under the eyelids is required (e.g., many patients are squeamish and/or fail to apply correct amounts of pharmaceuticals). Additionally, while not shown it should be understood that the treatment devices (e.g., 208(1)-208(N)) can include or be in communication with a user interface (like user interface 210) (e.g., buttons, keys, touch screen, or the like) for inputting manual information and/or instructions and can include other components for basic functions such as circuitry and/or wireless transducers. It should be understood that more configurations of one or more devices in different housings are possible (e.g., wearable, removably adhered, standalone device that the patient stands or sits by, or the like), but treatment devices in elements A-D are shown for ease of illustration and description. For example, the housings can have one or more differently sized and shaped openings to deliver light through.

For instance, the treatment devices shown in FIG. 5, elements A, B, and C can include one or more light sources (and may include one or more magnetic field sources) to provide light therapy. Parameters and/or dosages of the light therapy can be determined and/or configured to provide the greatest ability to interact with cellular components and influence cellular behavior for healing. The one or more light sources can each be configured to deliver individual portions of the light therapy according to one or more parameters (e.g., wavelength, intensity, power, timing, pulsation, and the like). The parameter(s) can be chosen based on the specific condition of the anterior segment, the location in the anterior segment of the condition, condition and/or symptom severity, patient characteristics, etc. For example, the parameter can be wavelength, which can correspond to light of different colors, with colors chosen from green, amber, blue, red, infrared, near-infrared etc. and the combination of colors (such that the treatment is optimized for effect on the condition being treated). The one or more light sources can operate according to one or more operational modes. The operational modes can include a pulsed operational mode, a continuous operational mode, and/or a superpulsed operational mode.

As an example, the treatment devices shown in FIG. 5, elements A, B, and C can include two or more light sources with different parameters (e.g., wavelength, therapy dose (including power, energy, exposure time, and the like), treatment frequency, etc.). Each of the light sources can be configured to deliver continuous light, pulsed light, and/or superpulsed light. As an example, wavelength can be the parameter varied between the two or more light sources in this example (such that the wavelengths correspond to a single wavelength or multiple different wavelengths) and each wavelength can have different therapeutic effects (however, the dose is also very important). The different wavelengths can cause different reactions at least because light at different wavelengths can be absorbed by specific chromophores within cells, initiating various photochemical reactions with outcomes ranging from enhanced cellular metabolism to modulation of signaling pathways, ultimately leading to therapeutic effects. The wavelengths can be selected independently and may be between 400 nm and 1100 nm. As an example, the wavelengths can be varied, and the wavelengths used can stimulate cellular activity, enhance tissue repair, modulate the inflammatory response, or the like. Longer wavelengths (e.g., red and infrared) can penetrate more deeply into tissues, stimulating mitochondrial activity and enhancing adenosine triphosphate (ATP) production, providing a source of energy crucial for cellular function and tissue repair. Additionally or alternatively, specific wavelengths and combinations can modulate immune responses and mitigate inflammation with multi-wavelength PBMT addressing different aspects of the inflammatory cascade. Additionally or alternatively, light in various wavelengths can influence intercellular signaling and gene expression with multi-wavelength approaches having the potential to target multiple signaling pathways leading to more nuanced and effective cellular communication. It should be noted that the optimal dose varies based on the specific condition, tissue depth, and individual patient.

The treatment devices shown in FIG. 5, elements A, B. and C can employ multiple wavelengths. Utilizing multiple wavelengths concurrently enables comprehensive and potent therapeutic effects by effectively targeting various cellular processes. Additionally, each wavelength possesses a unique tissue penetration depth and absorption profile, enhancing treatment across different tissue layers. This multi-wavelength approach also prevents the development of tolerance, avoiding reduced therapeutic effectiveness over time. Employing a variety of wavelengths ensures broader coverage of absorption spectra, eliminating “dead zones” where a single wavelength may be poorly absorbed. This adaptability allows for tailored treatment protocols, customized to specific conditions or desired therapeutic outcomes. Lastly, certain combinations of wavelengths have demonstrated synergistic effects, enhancing overall therapeutic benefits beyond what individual wavelengths can achieve. By leveraging the diversity of wavelengths, this approach optimizes cytochrome c oxidase activity, benefiting from spectral overlap and synergistic interactions. Simultaneously targeting multiple absorption peaks of cytochrome c oxidase enhances its activation and photochemical reactions, resulting in a more robust modulation of cellular metabolism and tissue repair processes through cumulative energy delivery from various wavelengths.

In some instances, the multiple light sources can include at least one pulsed light source or continuous light source and a super pulsed laser. For example, the light sources can include one or more groups of a super pulsed laser (emitting red and/or infrared light), a red diode (or multiple red diodes), and an infrared diode (or multiple infrared diodes), where at least a portion of the diodes can deliver pulsed light. The at least one pulsed light source and the super pulsed laser can each provide versatility in treatment. While not wishing to be bound by theory, it is believed that pulsing light is ideal for superficial treatment of the anterior segment as it minimizes tissue damage and promotes cell proliferation, collagen synthesis, and circulation to expedite healing. Light from super pulsed lasers can penetrate more deeply, stimulating cellular metabolism, reducing inflammation, and triggering repair mechanisms, while minimizing tissue risk. In fact, a combination of one or more super pulsed lasers, LED therapy, and magnetic fields can accelerate healing, inflammation control, and nutrient delivery, creating an optimal environment for tissue repair potentially by enhancing ATP production, metabolism, and cellular signaling. Indeed, immune modulation with PBM can help regulate IFN-γ levels, while allowing angiogenesis to promote tissue repair, addressing various aspects of the healing process.

Pulsing LEDs and lasers can emit light in a series of intermittent bursts. This approach minimizes the risk of thermal damage to surrounding tissues while allowing for precise control over the therapeutic dose. For example, it is believed that pulsing mode is particularly suitable for treating superficial corneal ulcerations by promoting cellular proliferation, collagen synthesis, and enhanced circulation. These ultimately accelerate the wound healing process.

Super pulsed lasers can emit high-energy bursts of light for ultra-short durations. This technology penetrates deeper into the tissues, making it ideal for treating deeper corneal ulcerations. The super pulse mode can stimulate cellular metabolism, reduce inflammation, and trigger a cascade of cellular repair mechanisms. By delivering energy in brief yet intense bursts, super pulsed lasers can maximize the therapeutic effects while minimizing the risk of tissue damage.

Static magnetic fields have been studied for their potential to influence cellular behavior and modulate various physiological processes. Without wishing to be bound by theory, it is believed that magnetic fields can affect ion movement, membrane potential, and cellular signaling. Additionally, static magnetic fields have shown the ability to enhance blood flow, which can be particularly beneficial in improving nutrient and oxygen delivery to ulcerated corneal tissues.

Super pulsed lasers and LED therapy, established for tissue regeneration and inflammation control, bring distinct advantages. Concurrently, static magnetic fields, known for their influence on cellular dynamics and vascular circulation, introduce an intriguing dimension to this therapeutic approach. This combined application of PBMT with super pulsed lasers and LEDs and static magnetic fields can expedite ulcer closure, regulate inflammation, and enhance nutrient delivery to the affected area. By synergizing these modalities, a conducive environment for tissue repair is envisioned, offering potential for more comprehensive and accelerated healing outcomes.

The treatment device shown in FIG. 5, element D is a specialized eye patch device (an eye-conforming LED patch is shown). The eye-conforming LED patch is a specialized device developed to address the challenges associated with delivering light energy to the eye effectively. Traditional methods often result in significant light energy loss due to factors like scattering, reflection, and inadequate contact with the ocular surface. This eye-conforming LED patch is designed to closely match the contours of the eye, ensuring that the emitted light energy is efficiently delivered to target areas. The eye-conforming LED patch is constructed using a flexible, biocompatible material that comfortably conforms to the curvature of the orbit, minimizing the gap between the light source and the orbit. This design feature ensures efficient light energy delivery, increasing the effectiveness of therapies and treatments. Two patches may be utilized simultaneously to cover both eyes. The eye-conforming LED patch includes an array of light-emitting diodes (LEDs) with specific wavelengths optimized for ocular applications, strategically positioned to provide even coverage and deliver the required therapeutic light energy to the eye. The eye-conforming LED patch can be powered by a battery source, making it highly portable and suitable for home use. Additionally, the eye-conforming LED patch can also be plugged into an external power supply for continuous operation, making it suitable for clinical and professional settings. The eye-conforming LED patch can contain a single wavelength or a combination of wavelengths, ranging from 400 to 1100 nm. It employs various light sources, including LEDs or lasers, which can emit light in either continuous or pulsed operation modes. The device may or may not include a static magnet.

Using any of the treatment devices shown in FIG. 5, elements A, B, C, and D, or otherwise known based off these illustrative examples, the effectiveness of treatment hinges on several critical factors, which encompass wavelength selection, energy dosage (dose), treatment frequency, and the presence of chromophores and porphyrins. Each emitted wavelength has distinct biological effects, underscoring the importance of choosing the right wavelength tailored to the specific condition. For instance, red light (600-700 nm) is known for its shallow penetration but has the capacity to reduce inflammation and trigger essential biochemical processes. On the other hand, near-infrared light (700-1100 nm) offers deeper tissue penetration, promoting enhanced circulation, pain reduction, and influencing cell membrane functions. Dose considerations in laser therapy encompass parameters such as power (in watts), energy (in joules), and exposure time (in seconds) to laser light. The optimal dose varies according to the condition, tissue depth, and individual patient characteristics. Therapeutic doses for laser therapy typically range from 0.001 to 10,000 joules (J), utilizing energy comprising single or multiple wavelengths spanning from 400 to 1100 nanometers (nm). Multiple light sources, including lasers and LEDs, can be employed in continuous, pulsed, or super-pulsed modes. The effectiveness of laser therapy is intricately linked to the selection of the appropriate wavelength and dose, which should align with the specific condition being treated. Dose requirements fluctuate based on variables such as wavelength composition, pulsing characteristics, light source selection, and the total energy delivered to the target tissue.

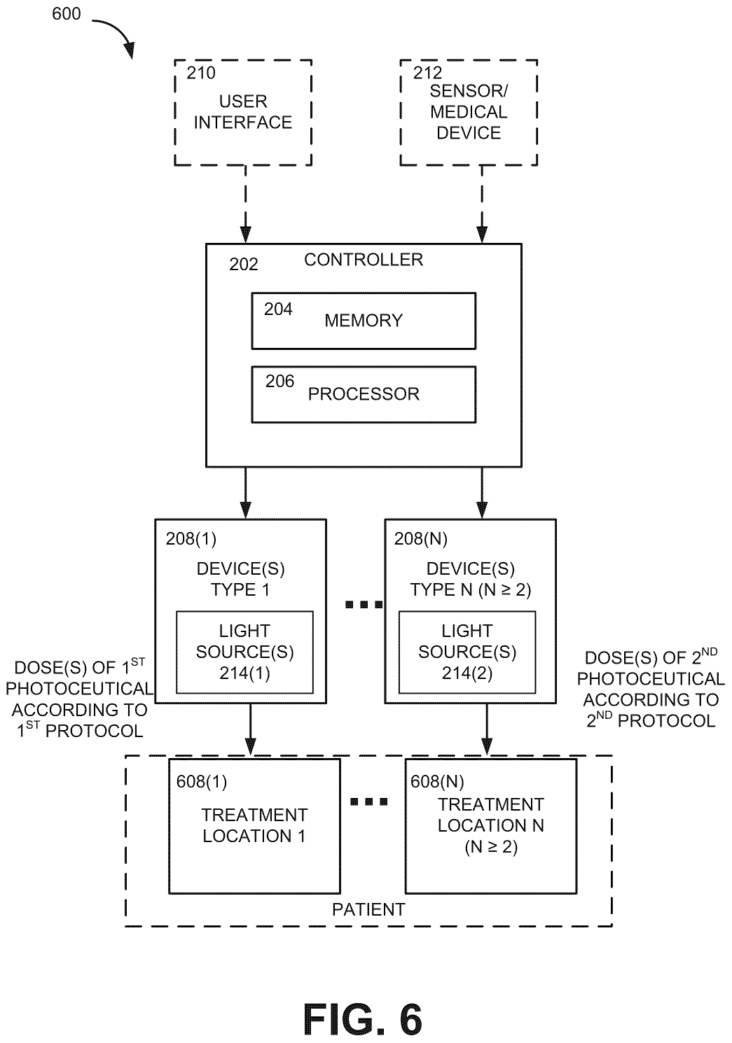

FIG. 6 is a diagram showing an example of the system 600 for developing a treatment plan for a patient suffering from another OSD and applying the treatment to the patient according to the treatment plan. The treatment plan of the system 600 includes two or more different treatment locations (e.g., treatment location 1 608(1)-treatment location N 608(N) with two or more photoceuticals and doses that can be applied according to different protocols. The controller 202 is the same as described with respect to FIGS. 2 and 3. A first photoceutical can be designed based on a first of the two treatment locations and the characteristic of the OSD. The first photoceutical comprises at least one first parameter (parameters described above). A second photoceutical can be designed based on a second of the two treatment locations and the characteristic of the OSD. The second photoceutical comprises at least one second parameter (parameters described above). A first treatment device 208(1) can be chosen to deliver at least one dose of the first photoceutical to the first of the two treatment locations. The first treatment device 208(1) can deliver the at least one dose of the first photoceutical using at least one light source 214(1). A second treatment device 208(N, N=2) can be chosen to deliver at least one dose of the second photoceutical to the second of the two treatment locations. The second treatment device 208(N, N=2) can deliver the at least one dose of the second photoceutical using at least one light source 214(N, N=2). The at least one dose of the first photoceutical and the at least one dose of the second photoceutical are configured based on the first of the two treatment locations and the second of the two treatment locations to work together to treat the OSD. As an example, the first of the two treatment locations can be on or near a portion of an eye having the OSD and/or near the eye having the OSD and the second of the two treatment locations can be remote from the eye having the OSD. For instance, the remote location can be a lymph node of the neck or groin, a thymus gland, a spleen, bone marrow in any location, or the like. In another instance the remote location can be a nerve such as the trigeminal nerve, a facial nerve, a cranial nerve, or the like).

### V. METHODS

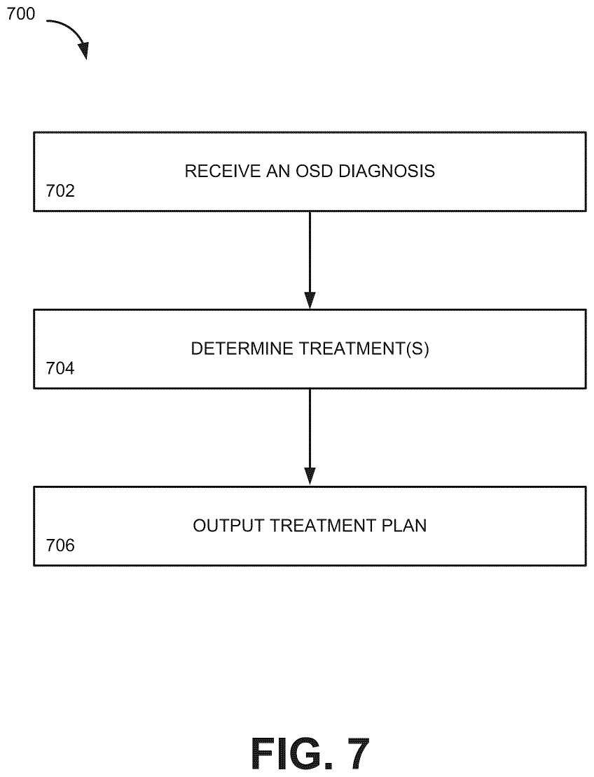

Another aspect of the present disclosure can include methods 700-900, as shown in FIGS. 7-9, for developing and applying a personalized treatment plan for an individual patient suffering from an ocular surface disorder (OSD). The personalized treatment plan can define one or more doses and dosage schedules of one or more photoceuticals to be applied to one or more treatment locations by one or more treatment devices. In some instances, the methods 700-900 can be executed by the controller of FIGS. 2, 3, and/or 6.

The methods 700-900 are illustrated as process flow diagrams with flowchart illustrations. For purposes of simplicity, the methods 700-900 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 700-900. Although a flowchart can describe the operations as a sequential process, many of the operations can be performed in parallel or concurrently. In addition, the order of the operations can be re-arranged. A process is terminated when its operations are completed but can have additional steps not included in the figure. A process can correspond to a method, a function, a procedure, a subroutine, a subprogram, etc. When a process corresponds to a function, its termination corresponds to a return of the function to the calling function or the main function.

One or more blocks of the respective flowchart illustrations, and combinations of blocks in the flowchart illustrations, can be implemented by computer program instructions. These computer program instructions can be stored in memory and provided to a processor of a general purpose computer, special purpose computer, and/or other programmable data processing apparatus to produce a machine, such that the instructions, which execute via the processor of the computer and/or other programmable data processing apparatus, create mechanisms for implementing the steps/acts specified in the flowchart blocks and/or the associated description. In other words, the steps/acts can be implemented by a system comprising a processor that can access the computer-executable instructions that are stored in a non-transitory memory.

Referring now to FIG. 7, illustrated is a method 700 for determining a treatment plan for a patient suffering from OSD. The treatment plan can include at least one noninvasive photoceutical. A configuration of the at least one photoceutical is described, but it should be known that other treatment types may be included in the treatment plan.

At 702, an OSD diagnosis can be received (e.g., input by a patient or caregiver associated with the patient, an employee of a clinic/doctor's office, input from a patient's electronic medical record (EMR), etc.). OSDs are a comprehensive category encompassing a diverse spectrum of conditions affecting at least a portion of a patient's anterior segment (e.g., the cornea, conjunctiva, related surfaces, and the like) and/or tear film. The conditions can include, but are not limited to, common discomforting issues like Dry Eye Disease (DED), Meibomian Gland Dysfunction (MGD), blepharitis, and conjunctivitis (pink eye) to more serious issues like corneal inflammation, abrasions, and ulcers, keratitis, Allergic Eye disease, recurrent corneal erosion, neurogenic eye pain, and neoplastic growths, such as Ocular Surface Squamous Neoplasia. OSD can also include ocular growths like Pterygium and Pinguecula, dermatological conditions such as rosacea, as well as complications stemming from glaucoma medications, chemical burns, thermal burns, immunological disorders such as Mucous Membrane Pemphigoid and Sjogren's Syndrome, and neurological based eye pain (e.g., trigeminal neuralgia, etc.). In some instances, OSDs can also include post-refractive eye surgery discomfort (specifically following procedures like LASIK, SMILE, and PRK, for example). In some instances, information about the location the OSD is affecting and/or specific symptom locations can also be received. Other information can include severity of the OSD and/or individual symptoms, previous treatments, patient demographic information, or the like. In other instances, one or more underlying or concurrent diagnoses can be received as well—such as diabetes, rheumatoid arthritis, Sjögren's syndrome, or Thyroid eye disease that may cause an OSD such as DED. In some instances, data from one or more sensors and/or medical devices can also be received that can be utilized to determine trending and/or real-time data related to the diagnosis and/or to amend a previous diagnosis.

At 704, one or more treatment plans can be determined. The one or more treatment plans can employ one or more non-invasive treatment options including applying one or more doses of one or more photoceuticals to one or more treatment locations. As an example, the one or more treatment locations can be associated with a patient's eye (e.g., ocular surface, tear film, meibomian gland, lacrimal gland, conjunctiva, corneal and/or optic nerve, or the like). However, the one or more treatment locations can include other parts of the patient's body (see, e.g., FIGS. 1 and 4 for bodily systems that may work in conjunction with and/or have an affect on the ocular surface) to maintain well-being of the ocular surface. For example, the other one or more treatment locations can include parasympathetic and/or sympathetic nerves of a patient, a thymus, a lymph node, a spleen, or the like. The photoceutical can include light therapy that can be delivered by a treatment device (example treatment devices shown in FIG. 5) in an amount to the one or more treatment locations according to a prescription and/or a configuration determined by the system (e.g., as described with respect to FIG. 2). The light therapy can include one or more light signals having one or more parameters (e.g., wavelength, intensity, power, timing, pulsation, and the like), intended to treat a certain condition. In some instances, the light therapy can combine a plurality of light signals into a photobiomodulation signal. In other instances, the light therapy may simply include one or more light signals that may be combined in different ways. A photoceutical may include application of one or more light signals at one or more doses from one or more light sources and one or more other light signals from one or more other light sources (where the light sources and the other light sources emit light having different wavelengths and/or intensities). The light sources can be LEDs, laser diodes, super-pulsed lasers, eye-safe lasers/light sources, or the like. The combination of light sources can provide one or more light signals with different properties to create a synergistic therapeutic effect that can treat one or more symptoms and/or underlying causes of the patient's OSD. For example, a therapy comprising at least one light signal at one wavelength and at least one other light signal at another wavelength can be emitted. However, the properties of the one or more light signals that are different are not limited to wavelengths, but could be treatment frequencies, powers, power densities, energies, energy densities, application times, exposure times, and/or other one or more properties. In some instances, the therapy can also include the emission of one or more additional light signals from one or more additional light sources that can have at least a different wavelength than the light signals from the light sources and the other light sources.

At 706, the treatment plan can be output. In some instances, the treatment plan can be output to the user (e.g., patient/someone caring for the patent, a doctor/employee associated with the doctor, or the like) and the user can program a treatment device according to the treatment plan. In other instances, the treatment plan can be output directly to the treatment device. The treatment plan can be used to treat the patient's OSD.

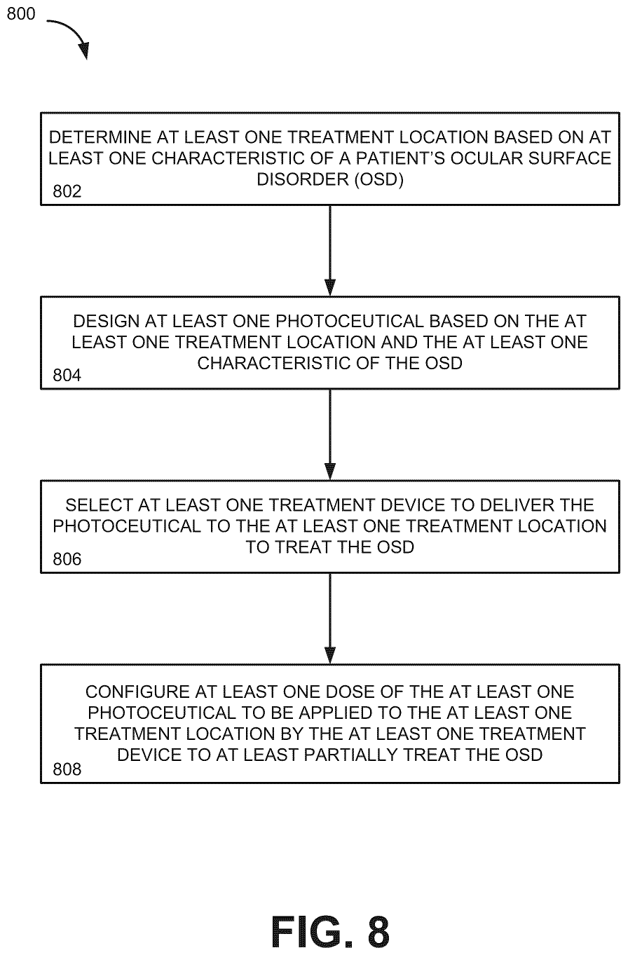

Referring now to FIG. 8, illustrated is a method 800 for defining features of the treatment plan. The features of the treatment plan can include at least one treatment location, at least one photoceutical, at least one treatment device, and at least one dose. The at least one dose of the at least one photoceutical can be designed to address interplay between at least two of inflammation, pain, and immune dysfunction related to the OSD.

At 802, at least one treatment location can be determined based on at least one characteristic of a patient's OSD. The characteristic of the OSD can include at least one of a type of the OSD, a condition of the OSD, a cause of the OSD, and at least one symptom of the OSD. In some instances, the at least one treatment location can include a portion of the patient's body that is local to the location of the eye suffering from OSD. In other instances, the at least one treatment location can include a portion of the patient's body that is remote from a location of an eye having the OSD. In still other instances, the at least one treatment location can include at least two treatment locations, including one or more portions of the patient's body that is local to the location of the eye suffering from OSD and one or more portions of the patient's body that is remote from a location of an eye having the OSD. In an example with two treatment locations, the first of the two treatment locations is a portion of an eye having the OSD and/or near the eye having the OSD and the second of the two treatment locations is remote from the eye having the OSD.

At 804, at least one photoceutical can be designed based on the at least one treatment location and the at least one characteristic of the OSD. The at least one photoceutical can include a parameter. As an example, the designing the photoceutical can include determining a photobiomodulation therapy comprising light of at least one wavelength emitted at least one light source. The at least one parameter of the at least one photoceutical can include: at least one wavelength of light capable of photobiomodulation, at least one magnetic field strength, at least a power or energy per dose, a pulsation characteristic, or the like. In the example above, the at least one dose of the first photoceutical and the at least one dose of the second photoceutical can be configured based on the first of the two treatment locations and the second of the two treatment locations to work together to treat the OSD.

At 806, at least one treatment device (e.g., one of the devices shown in FIG. 5A-D) can be selected to deliver the photoceutical to the at least one treatment location to treat the OSD. The selection can be based on the treatment location, the photoceutical, and/or the at least one characteristic of the OSD. As an example, the at least one treatment location comprises at least a portion of an ocular surface, one or more meibomian glands, one or more lacrimal glands, one or more nerves, one or more lymph nodes, etc. (e.g., any location shown in FIG. 1). The at least one treatment device can be chosen based on the at least one treatment location.

At 808, at least one dose of the at least one photoceutical can be configured to be applied to the at least one treatment location by the at least one treatment device. The at least one dose can include: a number of applications of the at least one photoceutical, a length of time of each application of the at least one photoceutical, an exposure time, a time between applications of the at least one photoceutical to the at least one treatment location, or the like.

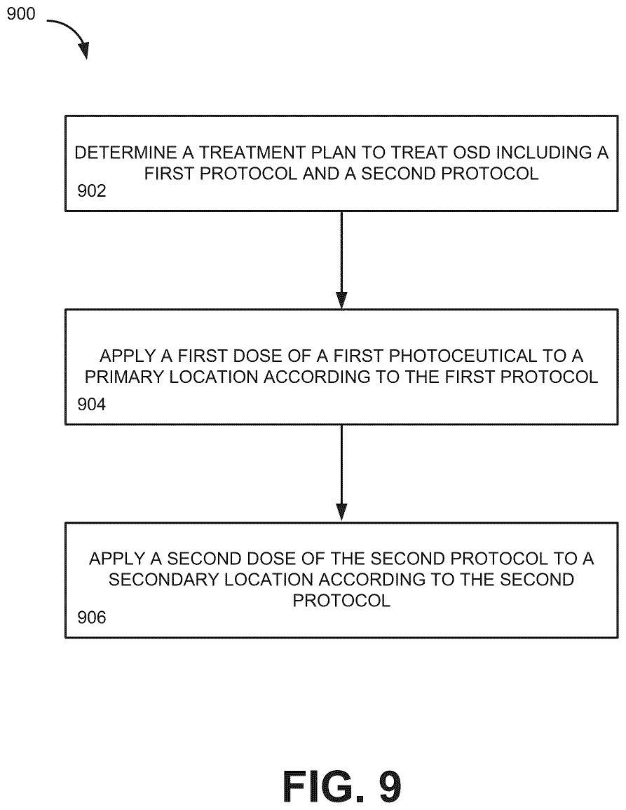

Referring now to FIG. 9, illustrated is a method 900 for applying at least two doses of light therapy to at least two locations on a patient's body according to a treatment plan. At 902, a first treatment plan can be determined to treat a patient's OSD, including a first protocol and a second protocol. The first protocol and the second protocol can include devices to apply photobiomodulation to the location on the patient's body. As an example, the first treatment device can be one of a handheld device, an eye-patch device, and a nasal application device and the second treatment device can be another of the handheld device, the eye patch device, and the nasal application device different from the first device. As an example, the at least one primary location comprises at least one of an ocular surface, a lacrimal gland, or a meibomian gland, while the at least one secondary location comprises at least one of at least one nerve or at least a portion of a lymphatic system. The at least the first photoceutical can be applied to the at least one primary location by a first treatment device and the at least the second photoceutical can be applied to the at least one secondary location by a second treatment device (the at least one primary location and the at least one secondary location are different). At 904, a first dose of a first photoceutical can be applied to a primary location according to a first protocol. At 906, a second dose of a second photoceutical can be applied to a secondary location according to a second protocol. The combination of at least the first photoceutical applied according to the first protocol and the at least the second photoceutical applied according to the second protocol improves a treatment outcome of a patient suffering from the OSD by addressing interplay between at least two of inflammation, pain, and immune dysfunction based in the at least one characteristic of the OSD.

### VI. EXAMPLES

Dry eye disease (DED) is an example of a common OSD with diverse physiological aspects involved. DED has various etiologies, manifestations, and associated factors, presenting as anywhere from mild to severe. When left untreated, DED can lead to significant discomfort and visual disturbances. DED encompasses both evaporative and aqueous forms, resulting from factors such as aging, environmental influences, medication side effects, lifestyle, or underlying medical conditions. It manifests in varying degrees of severity, from mild occasional discomfort to chronic and debilitating symptoms.

Addressing DED requires a multifaceted approach due to the diverse causes and the varied anatomical considerations involved. A combination of targets is essential as DED symptoms may stem from different physiological processes such as ocular surface inflammation, neuropathic pain, and immune system dysregulation. Patients often present with overlapping symptoms, and individual variability in responses mandates personalized interventions. A comprehensive healing process is crucial, considering the chronic and recurrent nature of DED. The combination of targets optimizes treatment outcomes by addressing multiple aspects simultaneously, offering a versatile and adaptable strategy for managing this complex ocular condition.

The systems and methods described above can be used to treat DED non-invasively using at least light therapy and aim to rejuvenate ocular tissues, alleviate inflammation, and expedite the healing process. Targeted treatments for each of the diverse physiological aspects involved in DED require precise customization of light parameters, including wavelengths and doses, to optimize therapeutic impact of this non-invasive therapy while minimizing the risks and other constraints that are normally seen with surgery, medication, and other current therapeutics. It should be noted that light parameters defining the treatment process are delineated within this protocol, including power density (e.g., between 0.0001 W/cm² and 100 W/cm²), energy density (e.g., between 0.001 J/cm² to 1000 J/cm²), pulsing frequencies (which can be continuous or pulse at frequencies from 1 Hz to 20,000 Hz), treatment duration (e.g., from 1 second to 60 minutes per session), and session frequency (e.g., sessions from 1 to 10 times per day). Safety considerations are paramount in these protocols, which emphasize patient comfort, avoidance of thermal damage, and preservation of ocular tissues.

Examples of how the systems and methods for developing a personalized treatment plan that is tailored for each patient suffering from DED are shown below. The following examples are not exhaustive. It should be noted that in real-world scenarios, a single patient may experience multiple physiological aspects of dry eye simultaneously contributing to their dry eye and one or more of the following solutions may be combined for each patient in a personalized and specific manner that accounts for any overlapping.

Condition: dry eye disease.

Physiological Aspect: inflammation on the ocular surface.

When a patient presents with inflammation on at least a portion of the ocular surface, the system executing the method for developing a patient-specific, non-invasive photoceutical based treatment plan (systems and methods described above) can determine that one or more doses of light therapy should be delivered to at least the portion of the ocular surface. It should be noted that the one or more doses of light therapy can be delivered to other parts of the ocular surface for treatment purposes as well (at the same or different times). The system can determine that the one or more doses of light therapy should be delivered by an eye patch device (e.g., similar to that shown in FIG. 5, element D) and/or using one or more handheld devices (e.g., similar to that shown in FIG. 5, element A) that can include eye-safe lasers or other light transmission devices according to a determined schedule. The schedule can be determined at least in part based on numerous factors including the level of severity of the inflammation, the specific location on the ocular surface of the inflammation, patient specific factors (e.g., sex, age, weight, other health conditions, etc.), or the like. Application of the light therapy to at least the portion of the ocular surface can cause the formation of anti-inflammatory agents, which can lead to the modulation of biological actions including localized inflammation. Accordingly, localized inflammation can be mitigated and/or resolved, thereby promoting a conducive environment for healing. Precise targeting of cytokines and chemokines is crucial for effective management.

Condition: dry eye disease.

Physiological Aspect: tear film production problem.

When a patient presents with a tear film production problem (e.g., meibomian gland dysfunction, lacrimal gland dysfunction, aqueous tear deficiency and tear instability, or the like), the system executing the method for developing a patient-specific, non-invasive photoceutical based treatment plan (systems and methods described above) may determine that one or more doses of light therapy should be delivered to one or more meibomian gland(s) and/or one or more lacrimal glands. The system can determine that the one or more doses of light therapy can be delivered by an eye patch device (e.g., similar to that shown in FIG. 5, element D) and/or a handheld device (e.g., similar to that shown in FIG. 5, element A) that can include one or more eye-safe lasers or other light transmission devices according to a certain schedule. The schedule can be determined at least in part based on numerous factors including the level of severity of the tear film production problem, which portions of the tear film are lacking, patient specific factors (e.g., sex, age, weight, other health conditions, etc.), or the like Application of light therapy to the one or more meibomian gland(s) and/or one or more lacrimal glands can improve tear composition (mucin, aqueous, and/or lipid layers) and/or improve the function of the meibomian glands and/or lacrimal glands to increase output, unblock the glands, or the like.

Condition: dry eye disease.

Physiological Aspect: neuropathic pain.

When a patient presents with neuropathic pain causing OSD or caused by OSD, the system executing the method for developing a patient-specific, non-invasive photoceutical based treatment plan (systems and methods described above) may determine that one or more doses of light therapy should be delivered by a device that goes through the nasal cavity (e.g., similar to that shown in FIG. 5, element C). The device can, for instance, include a light guide that can enable the light to pass through the nasal cavity without degradation. The light therapy can be delivered to the sphenopalatine ganglion (SPG) to cause a lessening of transmission of neuropathic pain signals to the brain. Light therapy can also or alternatively be delivered to the trigeminal nerve, the optic nerve, facial/cranial nerves, corneal nerves, and/or other nerves (e.g., through the nose, ear, or above the skin surface treatments depending on the depth of the nerve). Application of the light therapy, with distinct wavelengths and doses, can modulate nerve signaling and provide relief from symptoms, including pain and discomfort. In some instances, application of the light therapy can also reduce a sensation of dryness felt by the patient.

Condition: dry eye disease.

Physiological Aspect: immunological condition.

When a patient presents with an immunological condition causing or associated with an OSD (e.g., due to a systemic autoimmune disorder, such as Sjögren's syndrome, rheumatoid arthritis, lupus, and the like), the system executing the method for developing a patient-specific, non-invasive photoceutical based treatment plan (systems and methods described above) may determine that one or more doses of light therapy should be delivered to one or more locations on the user's body using by a handheld device for localized applications (e.g., similar to that shown in FIG. 5, element A) and/or a blanket type device for application to larger and/or more diverse locations on the body (e.g., similar to that shown in FIG. 5, element B). As an example, the location(s) can be one or more lymphoid organs and lymph node sites. For example, primary and secondary lymphoid organs can include the white pulp, lymph nodes, thymus gland, spleen, bone marrow, skin (dorsal and/or ventral), etc. The light delivery can prevent development of inflammation and regulate immune function by regulating immune factors throughout the whole body (as opposed to only localized regulation). In some instances, the light delivery (with specific wavelengths and doses) can also at least partially prevent the development of inflammation by modulating immune responses (striking a balance between immune suppression and maintaining ocular surface health).

From the above description, those skilled in the art will perceive improvements, changes and modifications. Such improvements, changes and modifications are within the skill of one in the art and are intended to be covered by the appended claims. All patents, patent applications, and publications cited herein are incorporated by reference in their entirety.

The following is claimed:

1. A method for determining a treatment plan for a patient suffering from an ocular surface disorder (OSD), the method comprising:
determining at least one treatment location based on at least one characteristic of the OSD;
designing at least one photoceutical based on the at least one treatment location and the characteristic of the OSD, wherein the at least one photoceutical comprises at least one parameter;
selecting at least one treatment device to deliver the photoceutical to the at least one treatment location to treat the OSD, wherein the at least one treatment device comprises at least one light source; and
configuring at least one dose of the at least one photoceutical to be applied to the at least one treatment location by the at least one treatment device to at least partially treat the OSD, wherein the at least one dose of the at least one photoceutical is designed to address interplay between at least two of inflammation, pain, and immune dysfunction related to the OSD.

2. The method of claim 1, wherein the characteristic of the OSD comprises at least one of a type of the OSD, a condition of the OSD, a cause of the OSD, and at least one symptom of the OSD.

3. The method of claim 1, wherein the at least one parameter of the at least one photoceutical comprises at least one of: at least one wavelength of light capable of photobiomodulation, at least one magnetic field strength, at least a power or energy per dose, and a pulsation characteristic.

4. The method of claim 1, wherein the at least one dose comprises at least one of: a number of applications of the at least one photoceutical, a length of time of each application of the at least one photoceutical, an exposure time, and/or a time between applications of the at least one photoceutical to the at least one treatment location.

5. The method of claim 1, wherein determining the at least one treatment location comprises determining two treatment locations, wherein the method further comprises:
designing a first photoceutical based on a first of the two treatment locations and the characteristic of the OSD, wherein the first photoceutical comprises at least one first parameter;
designing a second photoceutical based on a second of the two treatment locations and the characteristic of the OSD, wherein the second photoceutical comprises at least one second parameter;
choosing a first treatment device to deliver at least one dose of the first photoceutical to the first of the two treatment locations; and
choosing a second treatment device to deliver at least one dose of the second photoceutical to the second of the two treatment locations;
wherein the at least one dose of the first photoceutical and the at least one dose of the second photoceutical are configured based on the first of the two treatment locations and the second of the two treatment locations to work together to treat the OSD.

6. The method of claim 5, wherein the first of the two treatment locations is a portion of an eye having the OSD and/or near the eye having the OSD and the second of the two treatment locations is remote from the eye having the OSD.

7. The method of claim 1, wherein the at least one treatment location comprises a portion of the patient's body that is remote from a location of an eye having the OSD.

8. The method of claim 1, wherein the at least one treatment location comprises at least a portion of an ocular surface, one or more meibomian glands, one or more lacrimal glands, one or more nerves, and/or one or more lymph nodes.

9. The method of claim 1, wherein the designing the photoceutical further comprises determining a photobiomodulation therapy comprising light of at least one wavelength emitted at least one light source.

10. A method for treating an optical surface disorder (OSD), the method comprising:
determining a treatment plan to treat the OSD based on at least one characteristic of the OSD, the treatment plan comprising at least a first photoceutical and at least a second photoceutical;
applying at least one first dose of the at least the first photoceutical to at least one primary location according to a first protocol; and
applying at least one second dose of the at least the second photoceutical to at least one secondary location according to a second protocol,
wherein the combination of at least the first photoceutical applied according to the first protocol and the at least the second photoceutical applied according to the second protocol improves a treatment outcome of a patient suffering from the OSD by addressing interplay between at least two of inflammation, pain, and immune dysfunction based in the at least one characteristic of the OSD.

11. The method of claim 10, wherein the at least one primary location comprises at least one of an ocular surface, a lacrimal gland, or a meibomian gland.

12. The method of claim 10, wherein the at least one secondary location comprises at least one of at least one nerve or at least a portion of a lymphatic system.

13. The method of claim 10, wherein the at least the first photoceutical is applied to the at least one primary location by a first treatment device and the at least the second photoceutical is applied to the at least one secondary location by a second treatment device, wherein the at least one primary location and the at least one secondary location are different.

14. The method of claim 13, wherein the first treatment device is one of a handheld device, an eye-patch device, and a nasal application device and the second treatment device is another of the handheld device, the eye patch device, and the nasal application device different from the first device.

15. The method of claim 10, wherein the first protocol and the second protocol each comprise at least one parameter that is different from the other of the first protocol and the second protocol.

16. The method of claim 15, wherein the at least one parameter comprises at least one of at least one wavelength, at least one magnetic field strength, at least one pulsation property, a dose energy, an application time, and a number of applications.

17. The method of claim 10, wherein the at least one characteristic of the OSD comprises inflammation on an ocular surface, the at least one primary treatment location is an ocular surface to form localized anti-inflammatory agents to mitigate inflammation and promote a conducive environment for healing, and the at least one secondary treatment location is at least one of a lymphatic organ to regulate immune factors and at least partially prevent development of inflammation.

18. The method of claim 10, wherein the at least one characteristic of the OSD comprises meibomian gland dysfunction, the at least one primary treatment location comprises a meibomian gland and/or lacrimal gland to improve tear composition, and the at least one secondary treatment location is an ocular surface, a lymphatic organ, and/or a nerve to improve the pain, the inflammation, and/or the immune response associated with the OSD.

19. The method of claim 10, wherein the at least one characteristic of the OSD comprises neuropathic pain, the at least one primary treatment location comprises a sphenopalatine ganglion, a trigeminal nerve, an optic nerve, and/or a facial/cranial nerve to modulate nerve signaling and reduce pain, wherein the at least one secondary treatment location is an ocular surface and/or a lymphatic organ to improve the inflammation, and/or the immune response associated with the OSD

20. The method of claim 10, wherein the at least one characteristic of the OSD comprises an immunological cause, the at least one primary treatment location comprises at least one lymphatic organ, and the at least one secondary treatment location comprises at least another lymphatic organ to regulate immune factors and at least partially prevent development of inflammation.
